(19) United States
(12) Patent Application Publication
(10) Pub. No.: US 2024/0100369 A1
(43) Pub. Date: Mar. 28, 2024

(54) ELECTRONIC MASK AND METHOD OF CONTROLLING THE SAME

(71) Applicant: SAMSUNG ELECTRONICS CO., LTD., Suwon-si (KR)

(72) Inventors: Sungkoo Cho, Suwon-si (KR); Mukyong Kim, Suwon-si (KR); Mijo Kang, Suwon-si (KR); Kyungah Chang, Suwon-si (KR)

(73) Assignee: SAMSUNG ELECTRONICS CO., LTD., Suwon-si (KR)

(21) Appl. No.: 18/223,350

(22) Filed: Jul. 18, 2023

Related U.S. Application Data
(63) Continuation of application No. PCT/KR2023/009050, filed on Jun. 28, 2023.

(30) Foreign Application Priority Data
Sep. 22, 2022 (KR) 10-2022-0120166
Feb. 2, 2023 (KR) 10-2023-0014406

(51) Int. Cl. A62B 23/02; A62B 7/10

(52) U.S. Cl. CPC A62B 23/02; A62B 7/10

(57) ABSTRACT

Provided are an electronic mask and a method of controlling the electronic mask. The electronic mask includes at least one filter; at least one sensor configured to obtain sensing data related to the at least one filter; a fan configured to generate a flow of air toward the at least one filter; and at least one processor configured to: identify a grade of the at least one filter based on the sensing data obtained from the at least one sensor, and control a rotation speed of the fan based on the grade of the at least one filter.

### CROSS-REFERENCE TO RELATED APPLICATIONS

This application is a bypass continuation application of International Patent Application No. PCT/KR2023/009050, filed on Jun. 28, 2023, which is based on and claims priority to Korean Patent Application No. 10-2022-0120166, filed on Sep. 22, 2023 with the Korean Intellectual Property Office, and to Korean Patent Application No. 10-2023-0014406, filed on Feb. 2, 2023 with the Korean Intellectual Property Office, the disclosures of each of which are incorporated by reference herein in their entireties.

### BACKGROUND

### 1. Field

The disclosure relates to an electronic mask and a method of controlling the same, and more particularly, to a mask adapting in operation to a currently mounted filter.

### 2. Description of Related Art

Masks are for covering users' noses and mouths to prevent inhalation of foreign substances such as germs or dust. The mask may include an outer layer and an inner layer through which external air is filtered to be cleaned. The mask may include at least one filter that blocks foreign substances included in air introduced therein. For example, the at least one filter may include a high-efficiency particulate air (HEPA) filter that blocks fine dust. Accordingly, the mask may block foreign substances included in air to be introduced through the nose and mouth of the user when breathing.

Recently, an electronic mask for facilitating breathing of a user wearing the mask, including a filter, has been developed. The electronic mask may include a fan for introducing or discharging air to assist the user's breathing. In the electronic mask, at least one filter may be arranged adjacent to the fan. The electronic mask may block fine dust as much as possible through the at least one filter such that the user inhales clean air.

### SUMMARY

According to an aspect of the disclosure, an electronic mask includes: at least one filter; at least one sensor configured to obtain sensing data related to the at least one filter; a fan configured to generate a flow of air toward the at least one filter; and at least one processor configured to: identify a grade of the at least one filter based on the sensing data obtained from the at least one sensor, and control a rotation speed of the fan based on the grade of the at least one filter.

The at least one processor may be further configured to: control the at least one sensor to detect a signal transmitted from a radio-frequency identification (RFID) tag of the at least one filter, and identify the grade of the at least one filter based on the signal transmitted from the RFID tag.

The RFID tag may be provided on an edge region of the at least one filter, and may be configured to transmit the signal at a signal frequency in a predesignated frequency range, and the at least one sensor may be provided within a recognition distance of the RFID tag, the recognition distance being determined based on the predesignated frequency range.

The signal frequency may be between about 1 Hz and about 10 kHz, and the at least one sensor may be adjacent to the RFID tag to face the edge region of the at least one filter.

The signal frequency may be between about 1 MHz and about 10 GHz, and the at least one sensor may be provided on a control board to be spaced apart from the RFID tag, the control board including the at least one processor.

The at least one sensor may include a plurality of sensors, and the at least one processor may be further configured to: detect a shape of the at least one filter by obtaining a shape detection value related to the at least one filter from the plurality of sensors, and identify the grade of the at least one filter based on the shape of the at least one filter.

The plurality of sensors include: a first sensor provided in a first corner region of the at least one filter and configured to detect the shape of the at least one filter in a first direction, and a second sensor provided in a second corner region of the at least one filter and configured to detect the shape of the at least one filter in a second direction.

The at least one processor may be further configured to: detect a color of the at least one filter by obtaining a color detection value related to the at least one filter from the at least one sensor, and identify the grade of the at least one filter based on the color of the at least one filter.

The color may be applied to at least one pattern arranged on an edge region of the at least one filter, and the at least one sensor may be further configured to: output light toward the at least one pattern, and detect the color by detecting a reflected amount of the light.

The at least one processor may be further configured to increase the rotation speed of the fan as a dust collection efficiency related to the grade of the at least one filter mounted on the electronic mask increases.

The electronic mask may further include a communication interface configured to support wireless communication, and the at least one processor may be further configured to control the communication interface to: establish wireless communication with an external electronic device, and transmit data related to the electronic mask to the external electronic device.

The data related to the electronic mask may include the grade of the at least one filter and the rotation speed of the fan.

According to an aspect of the disclosure, a method of controlling an electronic mask including at least one filter, at least one sensor configured to obtain sensing data related to the at least one filter, and a fan configured to generate a flow of air toward the at least one filter, includes: identifying a grade of the at least one filter based on the sensing data obtained from the at least one sensor; and controlling a rotation speed of the fan based on the grade of the at least one filter.

The identifying of the grade of the at least one filter may include: detecting a signal transmitted from a radio-frequency identification (RFID) tag of the at least one filter; and identifying the grade of the at least one filter based on the signal transmitted from the RFID tag.

The at least one sensor may include a plurality of sensors, and the identifying of the grade of the at least one filter may include: detecting a shape of the at least one filter by obtaining a shape detection value related to the at least one filter from the plurality of sensors; and identifying the grade of the at least one filter based on the shape of the at least one filter.

The identifying of the grade of the at least one filter may include: detecting a color of the at least one filter by obtaining a color detection value related to the at least one filter from the at least one sensor; and identifying the grade of the at least one filter based on the color of the at least one filter.

The color may be applied to at least one pattern arranged on an edge region of the at least one filter, and the detecting of the color of the at least one filter may include: outputting light toward the at least one pattern, and detecting the color by detecting a reflected amount of the light.

The controlling of the rotation speed of the fan may include increasing the rotation speed of the fan as a dust collection efficiency related to the grade of the at least one filter mounted on the electronic mask increases.

The method may further include: establishing wireless communication with an external electronic device through a communication interface of the electronic mask; and transmitting data related to the electronic mask to the external electronic device through the communication interface.

The data related to the electronic mask may include the grade of the at least one filter and the rotation speed of the fan.

### BRIEF DESCRIPTION OF THE DRAWINGS

FIG. 1 is an exploded view diagram illustrating an electronic mask according to an embodiment of the disclosure.

FIG. 2 is a perspective view diagram illustrating a filter of an electronic mask according to an embodiment of the disclosure.

FIG. 3 is a diagram illustrating a clean air blowing module of an electronic mask according to an embodiment of the disclosure.

FIG. 4 is a diagram illustrating a clean air blowing module of an electronic mask according to an embodiment of the disclosure.

FIG. 5 is a diagram illustrating identification of the grade of a filter of an electronic mask based on the shape of the filter, according to an embodiment of the disclosure.

FIG. 6 is a diagram illustrating identification of the grade of a filter of an electronic mask based on the shape of the filter, according to an embodiment of the disclosure.

FIG. 7 is a diagram illustrating a clean air blowing module of an electronic mask according to an embodiment of the disclosure.

FIG. 8 is a perspective view diagram illustrating a filter of an electronic mask according to an embodiment of the disclosure.

FIG. 9 is a diagram illustrating progressions of light according to the color of a filter of an electronic mask according to an embodiment of the disclosure.

FIG. 10 is a diagram illustrating a clean air blowing module of an electronic mask according to an embodiment of the disclosure.

FIG. 11 is a diagram illustrating control of the rotation speed of a fan of an electronic mask according to an embodiment of the disclosure.

FIG. 12 is a diagram illustrating an electronic mask and an external electronic device, according to an embodiment of the disclosure.

FIG. 13 is a diagram illustrating an external electronic device displaying information about a filter of an electronic mask, according to an embodiment of the disclosure.

FIG. 14 is a diagram illustrating an external electronic device guiding a user to replace a filter of an electronic mask, according to an embodiment of the disclosure.

FIG. 15 is a diagram illustrating an external electronic device guiding a user to replace a filter of an electronic mask, according to an embodiment of the disclosure.

FIG. 16 is a block diagram illustrating an electronic mask according to an embodiment of the disclosure.

### DETAILED DESCRIPTION

Throughout the disclosure, the expression “at least one of a, b, or c” may indicate any of: only a, only b, only c, both a and b, both a and c, both b and c, or all of a, b, and c.

The terms used herein will be briefly described, and then an embodiment of the disclosure will be described in detail.

Although the terms used herein are selected from among common terms that are currently widely used in consideration of their functions in an embodiment of the disclosure, the terms may be different according to an intention of one of ordinary skill in the art, a precedent, or the advent of new technology. Also, in particular cases, the terms are discretionally selected by the applicant of the disclosure, in which case, the meaning of those terms will be described in detail in the corresponding description of an embodiment of the disclosure. Therefore, the terms used herein are not merely designations of the terms, but the terms are defined based on the meaning of the terms and content throughout the disclosure.

Throughout the disclosure, when a part “includes” an element, it is to be understood that the part may additionally include other elements rather than excluding other elements as long as there is no particular opposing recitation. In addition, as used herein, terms such as “ . . . er (or)”, “ . . . unit”, “ . . . module”, etc., denote a unit that performs at least one function or operation, which may be implemented as hardware or software or a combination thereof.

Hereinafter, embodiments of the disclosure will be described in detail with reference to the accompanying drawings to allow those of skill in the art to easily carry out the embodiments. An embodiment of the disclosure may, however, be embodied in many different forms and should not be construed as being limited to the embodiments of the disclosure set forth herein. Also, parts in the drawings unrelated to the detailed description are omitted to ensure clarity of the disclosure, and like reference numerals in the drawings denote like elements.

At least one filter included in an electronic mask may have various grades. Filters of different grades may have different air permeability. In a case in which rotation speed of a fan of the electronic mask is maintained at a specified value (which may be measured in revolutions per minute or rotations per minute (RPM)) regardless of the grade of the filter included in the electronic mask, even when a filter of an unsuitable grade is mounted on the electronic mask, the fan of the electronic mask may rotate at the specified RPM. When the fan of the electronic mask including the filter of the unsuitable grade rotates at the specified RPM, a user may experience discomfort in breathing. It is therefore desirable to control the rotation speed of the fan according to the grade of the at least one filter included in the electronic mask.

According to an embodiment of the disclosure, there may be provided an electronic mask capable of identifying the grade of at least one filter included in the electronic mask and allowing a user to easily identify the type and grade of the currently mounted filter, and a method of controlling the electronic mask.

According to an embodiment of the disclosure, there may be provided an electronic mask capable of controlling the rotation speed of a fan based on the grade of at least one filter, thereby reducing discomfort in breathing of a user even when a filter of a different grade is mounted on the electronic mask, and a method of controlling the electronic mask. The mask may thereby adapt in operation based on the currently mounted filter.

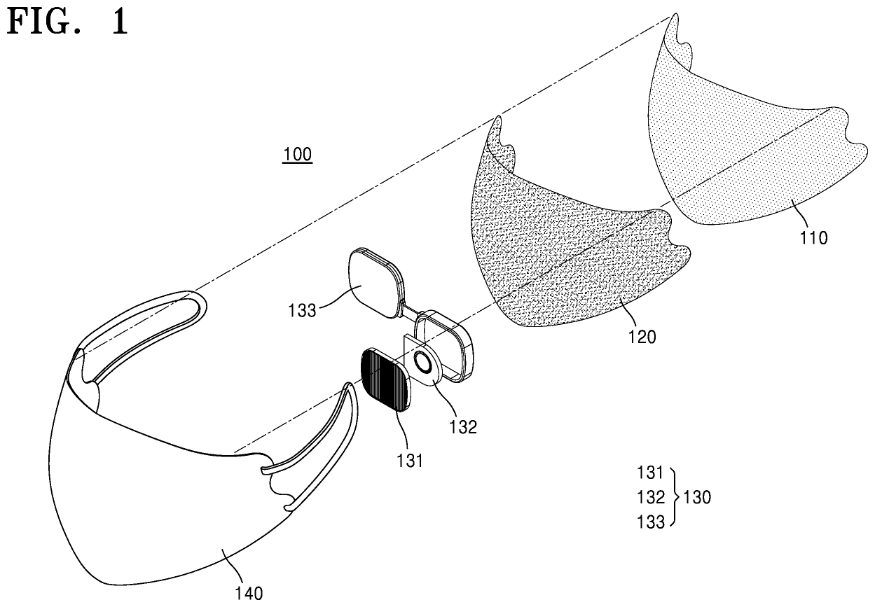

FIG. 1 is a diagram illustrating an electronic mask 100 according to an embodiment of the disclosure. The electronic mask 100 according to an embodiment of the disclosure may include a first inner layer 110, a second inner layer 120, a clean air blowing module 130, and an outer layer 140.

In an embodiment of the disclosure, the first inner layer 110 may be arranged to be adjacent to the front surface of the user's face including the nose and mouth. The first inner layer 110 may clean air to be inhaled by the user, before the air enters the user's nose and mouth. This cleaning may be a fine cleaning, relative to other cleanings provided by other elements of the mask 100. For example, the first inner layer 110 may block droplets contained in air exhaled by the user. For example, the first inner layer 110 may include a daily filter.

In an embodiment of the disclosure, the second inner layer 120 may be arranged in front of the first inner layer 110. The second inner layer 120 may clean the air to be inhaled by the user. This cleaning may be a coarser cleaning than that provided by the first inner layer 110. For example, the second inner layer 120 may block foreign substances such as germs or dust contained in the air. For example, the first inner layer 110 may include a Korea Filter (KF) filter.

In an embodiment of the disclosure, the outer layer 140 may be arranged on an outermost portion of the electronic mask 100. The outer layer 140 may pass external air to be inhaled by the user in a cleaner state. The outer layer 140 may form the exterior of the electronic mask 100. For example, the outer layer 140 may have various colors such as white, navy, or gray.

In an embodiment of the disclosure, the clean air blowing module 130 may smoothly introduce foreign substance-free air into the nose and mouth of the user wearing the electronic mask 100. The clean air blowing module 130 may include at least one filter 131, a fan 132, a case 133, and a driver circuit.

In an embodiment of the disclosure, the at least one filter 131 may be arranged in front of the second inner layer 120. The at least one filter 131 may have a round shape. The at least one filter 131 may clean air to be inhaled by the user. The at least one filter 131 may block fine dust contained in the air. For example, the at least one filter 131 may include a high-efficiency particulate air (HEPA) filter.

In an embodiment of the disclosure, the at least one filter 131 including the HEPA filter may be classified into various grades according to dust collection efficiency, inhalation resistance, total inward leakage, and the like. For example, the at least one filter 131 may be classified into various grades according to dust collection efficiency. The dust collection efficiency may be the proportion of fine dust collected and filtered out, from among fine dust contained in air. For example, the at least one filter 131 may have a Mask for Air cleaning (MA)-60 grade with a dust collection efficiency of 60% or greater, an MA-80 grade with a dust collection efficiency of 80% or greater, or an MA-94 grade with a dust collection efficiency of 94% or greater. As the dust collection efficiency of the at least one filter 131 increases, the amount of fine dust filtered out by the at least one filter 131 may increase. As the dust collection efficiency of the at least one filter 131 increases, breathing of the user wearing the electronic mask 100 may become more uncomfortable.

In an embodiment of the disclosure, the fan 132 may rotate to generate a flow of air toward the user's nose and mouth. The fan 132 may facilitate breathing of the user wearing the electronic mask 100. The fan 132 may be connected to a driver circuit included in the clean air blowing module.

In an embodiment of the disclosure, the driver circuit may supply power to rotate the fan 132. The driver circuit may be arranged inside the case 133. For example, the driver circuit may be mounted on an inner surface of the case 133. The driver circuit may be implemented as a printed board assembly (PBA). The driver circuit may include at least one processor and a communication module or communication interface.

In an embodiment of the disclosure, the at least one processor may include an arithmetic processing circuit for controlling the operation of the fan 132. For example, at least one processor may include a microcontroller (MCU). The at least one processor may identify the grade of the at least one filter 131. For example, the at least one processor may identify the grade of the HEPA filter included in the at least one filter 131. For example, the at least one processor may identify whether the at least one filter 131 corresponds to the MA-60 grade, the MA-80 grade, or the MA-94 grade. As such, the at least one processor may automatically identify the grade of the filter 131 mounted on the electronic mask 100. Accordingly, the user of the electronic mask 100 according to the disclosure may easily check the grade of the filter 131 mounted on the electronic mask 100.

In an embodiment of the disclosure, the at least one processor may control the rotation speed of the fan 132. The at least one processor may control the rotation speed of the fan 132 based on the grade of the at least one filter 131. As such, the at least one processor may automatically control the rotational speed of the fan 132 according to the grade of the at least one filter 131 mounted on the electronic mask 100. The clean air blowing module 130 of the electronic mask 100 according to an embodiment of the disclosure may provide a wearer of the electronic mask 100 with a flow of air suitable for the grade of the at least one filter 131. Accordingly, the electronic mask 100 according to an embodiment of the disclosure may reduce discomfort in the user's breathing even when a filter of a different grade is installed. That is, with any type of the filter 131 mounted on the electronic mask 100 by the user, the rotational speed of the fan 132 of the electronic mask 100 is controlled according to the grade of the filter 131, such that the user may comfortably breathe.

In an embodiment of the disclosure, the at least one processor may check the amount of charge of a battery included in the electronic mask 100. The at least one processor may control the rotation speed of the fan 132 based on the amount of charge of the battery. For example, the at least one processor may reduce the rotation speed of the fan 132 when the amount of charge of the battery is less than or equal to a specified threshold value. Accordingly, the at least one processor may manage the usage time of the battery by controlling the rotational speed of the fan 132 considering not only the grade of the at least one filter 131 but also the amount of charge of the battery.

In an embodiment of the disclosure, the communication module may establish a wireless communication connection with an external electronic device. For example, the communication module may establish a wireless communication connection with a mobile terminal such as a smart phone. The communication module may include a short-range wireless communication module for establishing a short-range wireless communication connection. For example, the communication module may include a Bluetooth Low Energy (BLE) communication module.

In an embodiment of the disclosure, the at least one processor may control the communication module to transmit data related to the electronic mask 100 to an external electronic device. For example, the at least one processor may transmit, to the external electronic device, the grade of the at least one filter 131 mounted on the electronic mask 100, the rotation speed of the fan 132 of the electronic mask 100, information about the remaining power of the battery included in the electronic mask 100, the number of breaths per minute of the user wearing the electronic mask 100, and information about the strength of flow of air passing through the at least one filter 131 to be introduced. Accordingly, the user of the electronic mask 100 may easily check information related to the electronic mask 100 by using the external electronic device.

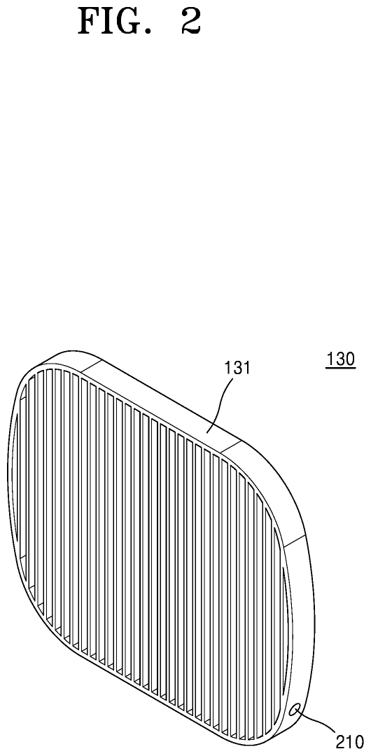

According to the disclosure, the grade of the filter 131 currently mounted on the electronic mask 100 worn by the user may be easily identified. The electronic mask 100 according to the disclosure may identify the grade of the at least one filter 131 mounted on the electronic mask 100, by using a sensing module. Hereinafter, a method of identifying the grade of the filter 131 by using the sensing module to which radio-frequency identification (RFID) technology is applied will be described with reference to FIG. 2.

FIG. 2 is a diagram illustrating the filter 131 of the electronic mask 100 according to an embodiment of the disclosure.

In an embodiment of the disclosure, the at least one filter 131 may include an RFID tag 210. Concave-convex patterns such as wrinkles may be formed on the front and rear regions of the at least one filter 131. An edge region of the at least one filter 131 may lack the concave-convex pattern. The RFID tag 210 may be arranged on the edge region of the at least one filter 131. For example, the RFID tag 210 may be arranged on a lateral edge region of the at least one filter 131.

In an embodiment of the disclosure, the RFID tag 210 may transmit a radio-frequency (RF) signal. The RF signal may include information about the at least one filter 131. For example, the RF signal may include information about the grade of the at least one filter 131, the type of the at least one filter 131, and whether the at least one filter 131 is mounted on the electronic mask 100. It is noted that the terms “radio-frequency” and “RF” are used herein for convenience, in light of the use of RFID technology, and the disclosure is not limited to frequencies understood to be radio frequency bands as the term is commonly understood. For example, use of transmission frequencies outside this range will be described later herein.

In an embodiment of the disclosure, a system to which the RFID technology is applied may include a reader part and a tag part. The reader part may recognize the tag part by receiving and processing a signal transmitted from the tag part. The reader part of the electronic mask 100 to which the RFID technology is applied may be the at least one processor. The tag part of the electronic mask 100 to which the RFID technology is applied may be the RFID tag 210.

In an embodiment of the disclosure, the at least one processor of the electronic mask 100 may recognize the RFID signal transmitted from the RFID tag 210 included in the at least one filter 131. The at least one processor may identify the grade of the at least one filter 131 based on the RF signal. The at least one processor may control the rotation speed of the fan 132 based on the identified grade of the at least one filter 131. The at least one processor may transmit identified information about the at least one filter 131 to the external electronic device.

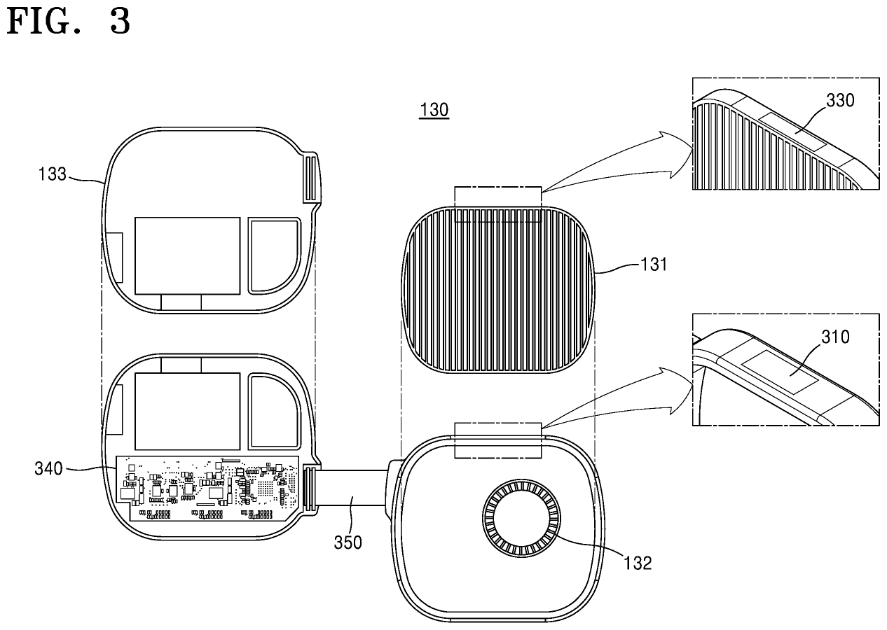

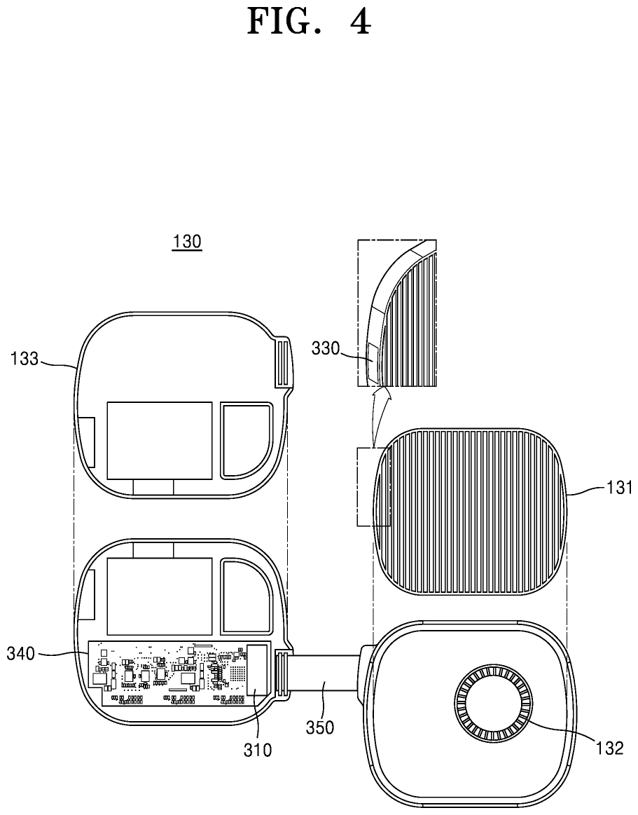

In a system to which the RFID technology is applied, when a reader part recognizes a tag part according to the frequency of an RF signal, a recognition distance between the reader part and the tag part may be determined. For example, as the frequency of the RF signal used in the system decreases, the recognition distance may decrease, and as the frequency of the RF signal increases, the recognition distance may increase. The recognition distance may therefore be determined for a given frequency or frequency range. A frequency range may be predesignated for RF signal use in the system. Accordingly, it is possible to determine how to implement the system to which the RFID technology is applied, based on the recognition distance of the predesignated frequency range. Hereinafter, the structures of the reader part and the tag part of the electronic mask 100 to which the RFID technology is applied will be described with reference to FIGS. 3 and 4. FIG. 3 may illustrate a structure in which the electronic mask 100 uses a low-frequency RF signal, and FIG. 4 may illustrate a structure in which the electronic mask 100 uses a high-frequency RF signal. It is noted that the terms “low-frequency” and “high-frequency”, as used herein, are defined relative to each other, and are not limited to the frequency ranges commonly described as “low-frequency” and “high-frequency” in either the context of RFID technology or the more general context of the radio spectrum. For example, use of transmission frequencies outside these ranges will be described later herein.

FIG. 3 is a diagram illustrating the clean air blowing module 130 of the electronic mask 100 according to an embodiment of the disclosure. The clean air blowing module 130 may include the at least one filter 131, the fan 132, and the case 133.

In an embodiment of the disclosure, the at least one filter 131 may be arranged inside the case 133. For example, the at least one filter 131 may be inserted into the case 133 to be adjacent to an inner surface of the case 133 on which the fan 132 is mounted. An RFID tag 330 may transmit a low-frequency RF signal at a signal frequency included in a first frequency range between about 1 Hz and about 10 kHz. The RF signal may include information about the at least one filter 131. For example, the RF signal may include information about the grade of the at least one filter 131.

In an embodiment of the disclosure, the at least one filter 131 may include the RFID tag 330. The RFID tag 330 may be arranged as a module at an edge region of the at least one filter 131. For example, the RFID tag 330 may be arranged on a lateral edge region of the at least one filter 131. The RFID tag 330 may be attached to the at least one filter 131. For example, the RFID tag 330 may be attached to an upper portion of the lateral edge region of the at least one filter 131, in the form of a film. However, the position of the RFID tag 330 is not limited thereto, and the RFID tag 330 may be attached to an arbitrary position on a lateral surface of the at least one filter 131.

In an embodiment of the disclosure, the fan 132 may be mounted on the inner surface of the case 133. For example, the fan 132 may be mounted on the rear surface of the case 133. The fan 132 may blow air toward the at least one filter 131.

In an embodiment of the disclosure, the case 133 may form an outer edge of the clean air blowing module 130. The case 133 may fix the at least one filter 131 and the fan 132 such that the at least one filter 131 and the fan 132 are arranged in designated positions and directions. The case 133 may include a sensing module 310, a control board 340, and a connector 350.

In an embodiment of the disclosure, the sensing module 310 may include at least one sensor. As the first frequency range, at which the RFID tag 330 transmits the low-frequency RF signal, has a relatively short recognition distance, the sensing module 310 may be arranged adjacent to the RFID tag 330. The sensing module 310 may be arranged to face an edge region of the at least one filter 131. The sensing module 310 may be a reader part for RFID technology. The sensing module 310 may obtain sensing data related to the at least one filter 131. The sensing module 310 may obtain the sensing data from the RFID tag 330. For example, the sensing module 310 may recognize a RF signal transmitted by the RFID tag 330 and obtain the sensing data based on the recognized RF signal. The sensing data may include information about the at least one filter 131. For example, the sensing data may include information about the grade of the at least one filter 131.

In an embodiment of the disclosure, the control board 340 may be arranged inside the case 133. For example, the control board 340 may be mounted on the inner surface of the case 133. The control board 340 may include at least one processor. The control board 340 may control the overall operation of the clean air blowing module 130 and the sensing module 310.

In an embodiment of the disclosure, when the case 133 is opened, the connector 350 may connect the left and right portions of the case 133 to each other. The connector 350 may electrically connect the control board 340 to the sensing module 310. For example, the connector 350 may include therein a wire that electrically connects the control board 340 to the sensing module 310.

FIG. 4 is a diagram illustrating the clean air blowing module 130 of the electronic mask 100 according to an embodiment of the disclosure. The clean air blowing module 130 may include the at least one filter 131, the fan 132, and the case 133.

In an embodiment of the disclosure, the at least one filter 131 may be arranged inside the case 133. For example, the at least one filter 131 may be inserted into the case 133 to be adjacent to the inner surface of the case 133 on which the fan 132 is mounted. The RFID tag 330 may transmit a high-frequency RF signal at a signal frequency included in a second frequency range between about 1 MHz and about 10 GHz. The RF signal may include information about the at least one filter 131. For example, the RF signal may include information about the grade of the at least one filter 131.

In an embodiment of the disclosure, the at least one filter 131 may include the RFID tag 330. The RFID tag 330 may be arranged as a module at an edge region of the at least one filter 131. For example, the RFID tag 330 may be arranged on a lateral edge region of the at least one filter 131. The RFID tag 330 may be attached to the at least one filter 131. For example, the RFID tag 330 may be attached to a left portion of the lateral edge region of the at least one filter 131, in the form of a film. However, the position of the RFID tag 330 is not limited thereto, and the RFID tag 330 may be attached to an arbitrary position on a lateral surface of the at least one filter 131.

In an embodiment of the disclosure, the fan 132 may be mounted on the inner surface of the case 133. For example, the fan 132 may be mounted on the rear surface of the case 133. The fan 132 may blow air toward the at least one filter 131.

In an embodiment of the disclosure, the case 133 may form an outer edge of the clean air blowing module 130. The case 133 may fix the at least one filter 131 and the fan 132 such that the at least one filter 131 and the fan 132 are arranged in designated positions and directions. The case 133 may include the sensing module 310, the control board 340, and the connector 350.

In an embodiment of the disclosure, the sensing module 310 may include at least one sensor. As the second frequency range, at which the RFID tag 330 transmits the high-frequency RF signal, has a longer recognition distance than the first frequency range, the sensing module 310 may be arranged to be spaced apart from the RFID tag 330. For example, the sensing module 310 may be arranged on the control board 340. In a case in which the sensing module 310 is arranged on the control board 340, the connector 350 need not include a connection wire, such as a wire, that electrically connects the control board 340 to the sensing module 310. However, the position of the sensing module 310 is not limited thereto, and the sensing module 310 may be arranged at an arbitrary position inside the case 133.

In an embodiment of the disclosure, the sensing module 310 may be a reader part for RFID technology. The sensing module 310 may obtain sensing data related to the at least one filter 131. The sensing module 310 may obtain the sensing data from the RFID tag 330. For example, the sensing module 310 may recognize a RF signal transmitted by the RFID tag 330 and obtain the sensing data based on the recognized RF signal. The sensing data may include information about the at least one filter 131. For example, the sensing data may include information about the grade of the at least one filter 131.

In an embodiment of the disclosure, the control board 340 may be arranged inside the case 133. For example, the control board 340 may be mounted on the inner surface of the case 133. The control board 340 may include at least one processor. The control board 340 may control the overall operation of the clean air blowing module 130 and the sensing module 310.

In an embodiment of the disclosure, when the case 133 is opened, the connector 350 may connect the left and right portions of the case 133 to each other.

Herein, for convenience, the control board 340 and the at least one processor will sometimes be described interchangeably, when functions specific to a processor are being described. It will be appreciated by those of skill in the art that the same or similar functions may be implemented by components other than a processor in other embodiments, and that the disclosure is therefore not limited thereto.

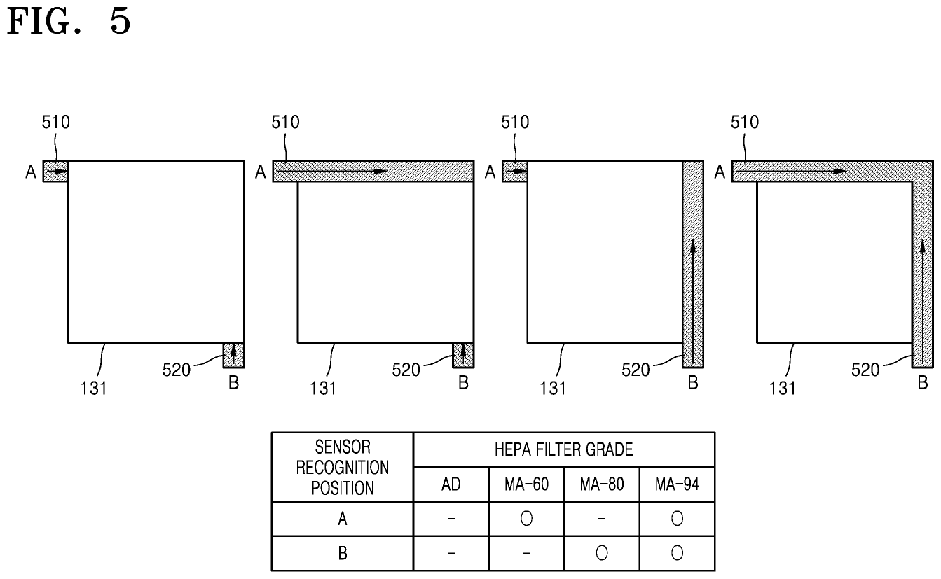

The shape of the at least one filter 131 may be determined according to the grade of the at least one filter 131. Hereinafter, a method of identifying the grade of the filter 131 based on the shape of the filter will be described with reference to FIGS. 5 to 7.

FIG. 5 is a diagram illustrating identification of the grade of the filter 131 of the electronic mask 100 based on the shape of the filter 131, according to an embodiment of the disclosure.

In an embodiment of the disclosure, a sensing module may include, or be coupled to, a plurality of sensors 510 and 520. The plurality of sensors 510 and 520 may be arranged at different positions. The plurality of sensors 510 and 520 may detect the shape of the at least one filter 131 in different directions. For example, each of the plurality of sensors 510 and 520 may include an optical sensor, an ultrasonic sensor, a microwave sensor, an infrared sensor, a touch sensor, or a light-emitting diode (LED) sensor. For example, each of the plurality of sensors 510 and 520 may include a limit switch, a micro switch, and a push switch. For example, the plurality of sensors 510 and 520 may include a first sensor 510 and a second sensor 520.

In an embodiment of the disclosure, the first sensor 510 may be arranged in a first corner region of the at least one filter 131. For example, the first sensor 510 may be arranged in an upper-left corner region of the at least one filter 131. The first sensor 510 may detect the shape of the at least one filter 131, in a first direction. For example, the first sensor 510 may detect the shape of the at least one filter 131, starting from the left end of the at least one filter 131 toward the right end.

In an embodiment of the disclosure, the second sensor 520 may be arranged in a second corner region of the at least one filter 131. The second corner region may be a corner region different from the first corner region. For example, the second corner region may be a corner region located diagonally opposite to the first corner region with respect to the at least one filter 131. For example, the second sensor 520 may be arranged in a lower-right corner region of the at least one filter 131. The second sensor 520 may detect the shape of the at least one filter 131, in a second direction. The second direction may be different from the first direction. For example, the second direction may be perpendicular or substantially perpendicular to the first direction. For example, the second sensor 520 may detect the shape of the at least one filter 131, starting from the lower end of the at least one filter 131 toward the upper end.

In an embodiment of the disclosure, the at least one processor 340 may obtain a shape detection value related to the at least one filter 131, from the plurality of sensors 510 and 520. The at least one processor 340 may control the plurality of sensors 510 and 520 to detect the shape of the at least one filter 131. The at least one processor 340 may identify whether an edge of the at least one filter 131 is detected at the position of each of the plurality of sensors 510 and 520. The at least one processor 340 may obtain sensing data about a position recognized as an edge of the at least one filter 131, from each of the plurality of sensors 510 and 520. For example, the at least one processor 340 may obtain first position information A about a position recognized as an edge, from the first sensor 510. For example, the at least one processor 340 may obtain second position information B about a position recognized as an edge, from the second sensor 520. For example, when either there is no position recognized as an edge by the first sensor 510, or a position recognized as an edge by the first sensor 510 has a length less than or equal to a specified threshold value, the at least one processor 340 may determine that the first position information A does not exist (-). For example, when a position recognized as an edge by the first sensor 510 has a length greater than the specified threshold value, the at least one processor 340 may determine that the first position information A exists (O). For example, when either there is no position recognized as an edge by the second sensor 520, or a position recognized as an edge by the second sensor 520 has a length less than or equal to the specified threshold value, the at least one processor 340 may determine that the second position information B does not exist (-). For example, when a position recognized as an edge by the second sensor 520 has a length greater than the specified threshold value, the at least one processor 340 may determine that the second position information B exists (O).

In an embodiment of the disclosure, the at least one processor 340 may determine the shape of the at least one filter 131 based on the sensing data obtained from each of the plurality of sensors 510 and 520. The at least one processor 340 may determine the shape of an edge of the at least one filter 131 based on position information (e.g., A and B) obtained from each of the plurality of sensors 510 and 520. For example, when the first position information A does not exist (-) and the second position information B does not exist (-), the at least one processor 340 may determine that the at least one filter 131 has approximately the shape of a square with a short edge. For example, when the first position information A exists (O) and the second position information B does not exist (-), the at least one processor 340 may determine that the at least one filter 131 has approximately the shape of a rectangle with vertical sides longer than horizontal sides. For example, when the first position information A does not exist (-) and the second position information B exists (O), the at least one processor 340 may determine that the at least one filter 131 has approximately the shape of a rectangle with horizontal sides longer than vertical sides. For example, when the first position information A exists (O) and the second position information B exists (O), the at least one processor 340 may determine that the at least one filter 131 has approximately the shape of a square with a long edge.

In an embodiment of the disclosure, the at least one processor 340 may identify the grade of the at least one filter 131 based on the shape of the at least one filter 131. The at least one processor 340 may determine the grade of the at least one filter 131 to correspond to the shape of the at least one filter 131. The at least one processor 340 may identify the grade of the at least one filter 131 based on the position information A and B respectively obtained from the plurality of sensors 510 and 520. For example, when the first position information A does not exist (-) and the second position information B does not exist (-), the at least one processor 340 may determine that the grade of the at least one filter 131 is an AD grade. For example, when the first position information A exists (O) and the second position information B does not exist (-), the at least one processor 340 may determine that the grade of the at least one filter 131 is an MA-60 grade. For example, when the first position information A does not exist (-) and the second position information B exists (O), the at least one processor 340 may determine that the grade of the at least one filter 131 is an MA-80 grade. For example, when the first position information A exists (O) and the second position information B exists (O), the at least one processor 340 may determine that the grade of the at least one filter 131 is an MA-94 grade. As such, the at least one processor 340 may recognize the shape of the at least one filter 131, and easily identify the grade of the at least one filter 131 based on the recognized shape.

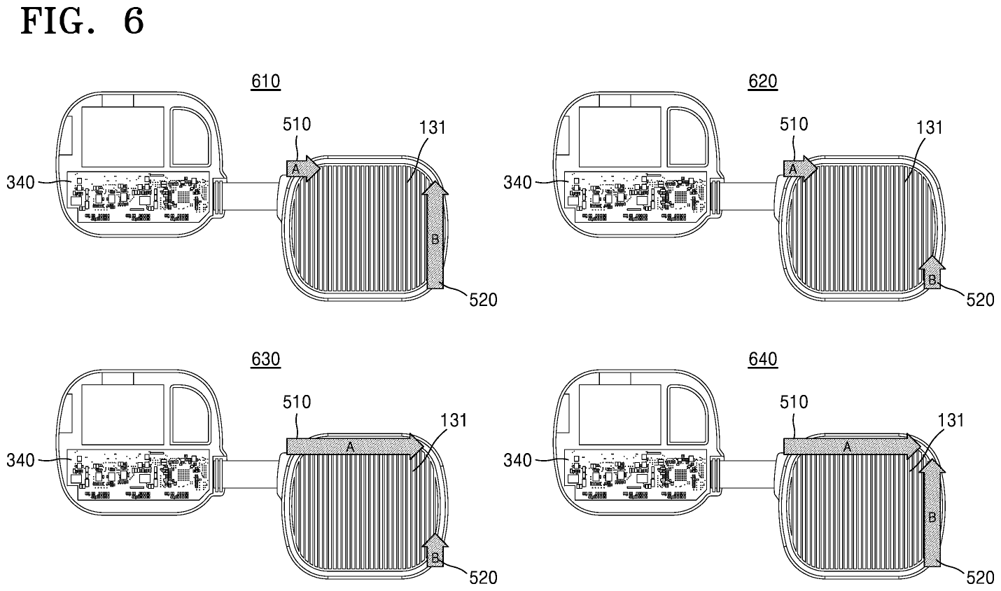

FIG. 6 is a diagram illustrating identification of the grade of the filter 131 of the electronic mask 100 based on the shape of the filter 131, according to an embodiment of the disclosure.

In an embodiment of the disclosure, the at least one processor 340 may recognize the shape of the at least one filter 131 based on sensing data obtained from the plurality of sensors 510 and 520. For example, the at least one processor 340 may detect the shape of the at least one filter 131 based on the first position information A obtained from the first sensor 510 and the second position information B obtained from the second sensor 520.

In an embodiment of the disclosure, the first sensor 510 may be arranged at a first position and the second sensor 520 may be arranged at a second position. For example, the first sensor 510 may be arranged on an upper left portion of the case 133. For example, the second sensor 520 may be arranged on a lower right portion of the case 133. However, the disclosure is not limited thereto, and the first sensor 510 and the second sensor 520 may be arranged at arbitrary positions in the case 133 to which the at least one filter 131 is coupled. For example, at least one of the first sensor 510 or the second sensor 520 may be arranged on a fixing part of the case 133. The fixing part of the case 133 may be a part that couples the at least one filter 131 to the case 133 such that the at least one filter 131 is attached to the inside of the case 133.

In an embodiment of the disclosure, the at least one processor 340 may classify the shape of the at least one filter 131 into four grades, based on the first position information A obtained from the first sensor 510 and the second position information B obtained from the second sensor 520. The at least one processor 340 may identify the grade of the at least one filter 131 by matching each of the four types of filter to each of the four grades on a one-to-one basis. For example, when the first position information A does not exist (-) and the second position information B does not exist (-) as in a first situation 610, the at least one processor 340 may determine that the grade of the at least one filter 131 is the AD grade. For example, when the first position information A exists (O) and the second position information B does not exist (-) as in a second situation 620, the at least one processor 340 may determine that the grade of the at least one filter 131 is the MA-60 grade. For example, when the first position information A does not exist (-) and the second position information B exists (O) as in a third situation 630, the at least one processor 340 may determine that the grade of the at least one filter 131 is the MA-80 grade. For example, when the first position information A exists (O) and the second position information B exists (O) as in a fourth situation 640, the at least one processor 340 may determine that the grade of the at least one filter 131 is the MA-94 grade.

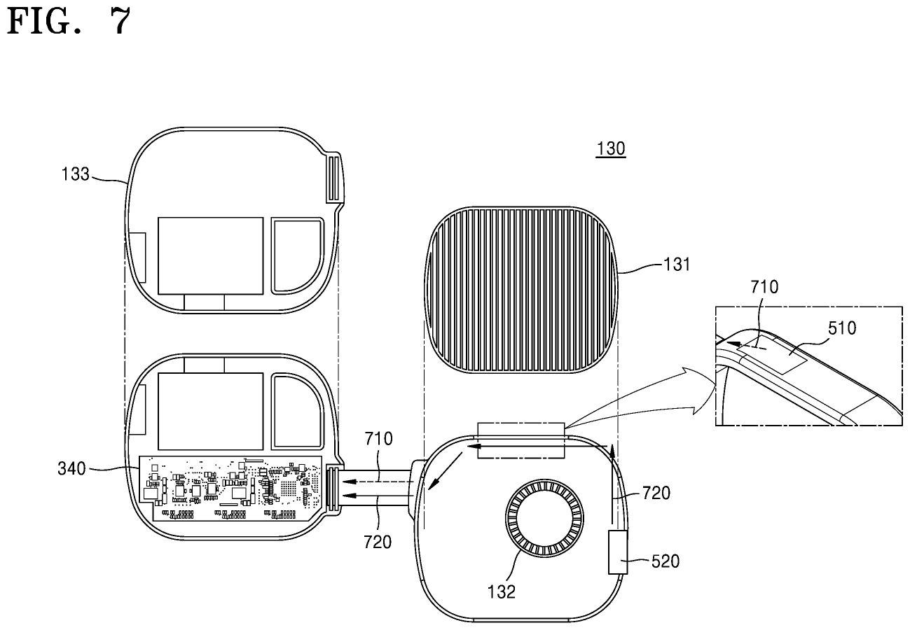

FIG. 7 is a diagram illustrating the clean air blowing module 130 of the electronic mask 100 according to an embodiment of the disclosure. The clean air blowing module 130 may include the at least one filter 131, the fan 132, and the case 133.

In an embodiment of the disclosure, the at least one filter 131 may be arranged inside the case 133. For example, the at least one filter 131 may be inserted into the case 133 to be adjacent to the inner surface of the case 133 on which the fan 132 is mounted.

In an embodiment of the disclosure, the fan 132 may be mounted on the inner surface of the case 133. For example, the fan 132 may be mounted on the rear surface of the case 133. The fan 132 may blow air toward the at least one filter 131.

In an embodiment of the disclosure, the case 133 may form an outer edge of the clean air blowing module 130. The case 133 may fix the at least one filter 131 and the fan 132 such that the at least one filter 131 and the fan 132 are arranged in designated positions and directions. The case 133 may include the control board 340 and the plurality of sensors 510 and 520.

In an embodiment of the disclosure, the control board 340 may be arranged inside the case 133. For example, the control board 340 may be mounted on the inner surface of the case 133. The control board 340 may include at least one processor. The control board 340 may control the overall operation of the clean air blowing module 130 and the plurality of sensors 510 and 520.

In an embodiment of the disclosure, the plurality of sensors 510 and 520 may include the first sensor 510 arranged at a first position and the second sensor 520 arranged at a second position. For example, the first sensor 510 may be arranged on an upper edge of the case 133. For example, the second sensor 520 may be arranged on a right edge of the case 133. However, the disclosure is not limited thereto, and the first sensor 510 and the second sensor 520 may be respectively arranged at two different points on an edge of the case 133.

In an embodiment of the disclosure, a first connection wire 710 may electrically connect the control board 340 to the first sensor 510. For example, a second connection wire 720 may be a wire arranged inside the case 133 to electrically connect the control board 340 to the first sensor 510.

In an embodiment of the disclosure, the second connection wire 720 may electrically connect the control board 340 to the second sensor 520. For example, the second connection wire 720 may be a wire arranged inside the case 133 to electrically connect the control board 340 to the second sensor 520.

In an embodiment of the disclosure, each of the plurality of sensors 510 and 520 may generate a signal is recognizable by the at least one processor 340 during operation. For example, when the first sensor 510 recognizes the at least one filter 131, the first sensor 510 may generate a high signal. For example, when the second sensor 520 recognizes the at least one filter 131, the second sensor 520 may generate a high signal. The at least one processor 340 may recognize at least one sensor having generated a signal among the plurality of sensors 510 and 520. The at least one processor 340 may identify the shape of the at least one filter 131 based on the signal generated by each of the plurality of sensors 510 and 520.

In an embodiment of the disclosure, in a case in which each of the plurality of sensors 510 and 520 is an optical sensor, the at least one processor 340 may identify the shape of the at least one filter 131 based on whether light output from a light emitting unit of each of the plurality of sensors 510 and 520 is blocked. In a case in which each of the plurality of sensors 510 and 520 is an optical sensor, a light emitting unit of each of the plurality of sensors 510 and 520 may output light toward an edge region of the at least one filter 131. Depending on the shape of the at least one filter 131, the light output from the light emitting unit of each of the plurality of sensors 510 and 520 may be selectively blocked. A light receiving unit may be arranged opposite the light emitting unit of each of the plurality of sensors 510 and 520. Each of the plurality of sensors 510 and 520 may generate a signal based on whether the light receiving unit receives the light output from the light emitting unit. For example, each of the plurality of sensors 510 and 520 may generate a high signal when the light is not received by the light receiving unit. The at least one processor 340 may recognize at least one sensor having generated a high signal among the plurality of sensors 510 and 520. The at least one processor 340 may determine that the light output from the light emitting unit is blocked by the at least one filter 131 in a region in which the at least one sensor having generated a high signal is located. The at least one processor 340 may identify the grade of the at least one filter 131 based on the region in which the light output from the light emitting unit is blocked.

In an embodiment of the disclosure, in a case in which each of the plurality of sensors 510 and 520 includes a switch, the at least one processor 340 may detect whether the switch included in each of the plurality of sensors 510 and 520 is pressed and thus turned on. When the at least one filter 131 is mounted on the case 133, the switch of each of the plurality of sensors 510 and 520 may be pressed by an edge region of the at least one filter 131. When the switch is turned on, each of the plurality of sensors 510 and 520 may generate a signal. For example, when the switch is turned on, each of the plurality of sensors 510 and 520 may generate a high signal. The at least one processor 340 may identify the grade of the at least one filter 131 based on the signal generated when the switch of each of the plurality of sensors 510 and 520 is pressed and turned on.

In an embodiment of the disclosure, in a case in which each of the plurality of sensors 510 and 520 is an electrostatic sensor, the at least one processor 340 may detect a capacitance value detected by each of the plurality of sensors 510 and 520. The capacitance value detected by each of the plurality of sensors 510 and 520 may be determined according to the shape of the at least one filter 131 mounted on the case 133. For example, the capacitance values respectively detected by the plurality of sensors 510 and 520 may vary depending on whether the edge region of the at least one filter 131 covers the plurality of sensors 510 and 520. The at least one processor 340 may compare the detected capacitance value with a capacitance value before the at least one filter 131 is mounted on the case 133, to obtain the amount of change in capacitance value after the at least one filter 131 is mounted on the case 133, from each of the plurality of sensors 510 and 520. The at least one processor 340 may identify the shape of the at least one filter 131 based on the amount of change in the capacitance value obtained from each of the plurality of sensors 510 and 520, to identify the grade of the at least one filter 131. The at least one filter 131 may include a counterpart such as a metal body in order to increase the amount of change in the capacitance value.

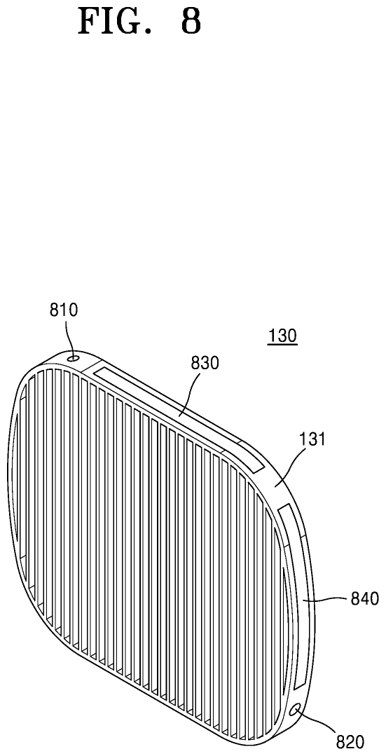

Hereinafter, a method of identifying the grade of the filter 131 based on the color of the filter 131 will be described with reference to FIGS. 8 to 10.

FIG. 8 is a diagram illustrating the filter 131 of the electronic mask 100 according to an embodiment of the disclosure.

In an embodiment of the disclosure, the at least one filter 131 may include at least one pattern; for example, first to fourth patterns 810, 820, 830, and 840. The patterns 810, 820, 830, and 840 may be arranged on an edge region of the at least one filter 131. The patterns 810, 820, 830, and 840 may be arranged in a dot shape or in a shape extending along an edge of the at least one filter 131. For example, among the patterns 810, 820, 830, and 840, the first pattern 810 and the second pattern 820 may be arranged in a dot shape, and the third pattern 830 and the fourth pattern 840 may be arranged in a quadrangular shape extending along the edge of the at least one filter 131.

In an embodiment of the disclosure, designated colors may be applied to the patterns 810, 820, 830, and 840, respectively. For example, the inside of each of the patterns 810, 820, 830, and 840 may be colored with a designated color. The color may be any color with a luminance distinguishable according to brightness. For example, red may be applied to the first pattern 810 and the third pattern 830 and blue may be applied to the second pattern 820 and the fourth pattern 840, among the patterns 810, 820, 830, and 840. The color applied to each of the patterns 810, 820, 830, and 840 may be determined according to the grade of the at least one filter 131. In a case in which the patterns 810, 820, 830, and 840 are arranged in an edge region of the at least one filter 131 in a dot shape or a shape extending along an edge according to the grade of the at least one filter 131, and the patterns 810, 820, 830, and 840 are colored with colors determined according to the grade of the at least one filter 131 as described above, the at least one processor 340 may obtain a color detection value related to the at least one filter 131, from the sensing module 310. For example, the at least one processor 340 may detect the colors of the patterns 810, 820, 830, and 840 by using a proximity sensor or a color sensor, and identify the grade of the at least one filter 131 based on the detected colors.

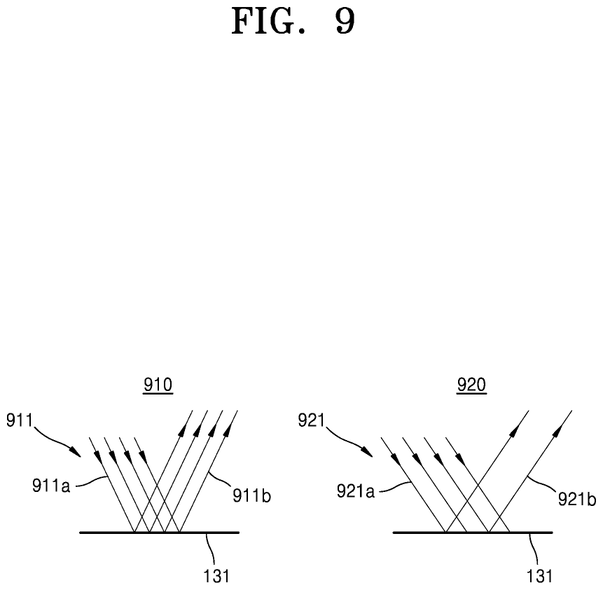

FIG. 9 is a diagram illustrating progressions of light 911 and 921 according to the color of the filter 131 of the electronic mask 100 according to an embodiment of the disclosure.

In an embodiment of the disclosure, the patterns 810, 820, 830, and 840 on the surface of the at least one filter 131 of the electronic mask 100 may be colored with determined colors. For example, the patterns 810, 820, 830, and 840 may be arranged in an edge region of the at least one filter 131 in a dot shape or a shape extending along an edge, and colored with colors determined according to the grade of the at least one filter 131.

In an embodiment of the disclosure, the progressions of the light 911 and 921 may be determined according to the colors of each of the patterns 810, 820, 830, and 840 of the at least one filter 131. For example, the reflectance for the light 911 or 921 may be determined according to the colors of the patterns 810, 820, 830, and 840. In a first situation 910, most of the incident light 911a may be reflected as reflected light 911b. In a second situation 920, a part of the incident light 921a may be absorbed and the rest may be reflected as reflected light 921b. The at least one processor 340 may detect the colors of the patterns 810, 820, 830, and 840 by measuring the amounts of the reflected light 911b and 921b compared to the amounts of the incident light 911a and 921a by using a proximity sensor or a color sensor.

In an embodiment of the disclosure, the proximity sensor or color sensor may output light toward the surface of a target object, and when the output light is reflected from the surface of the target object, determine the amount of light that has been reflected and returned to the sensor, to detect the color of the target object. For example, in order to detect the colors of the patterns 810, 820, 830, and 840 arranged on the edge region of the at least one filter 131, the proximity sensor or color sensor may output light toward the patterns 810, 820, 830, and 840 and determine the amount of light that has been reflected and returned to the sensor.

In an embodiment of the disclosure, in a case in which the colors of each of the patterns 810, 820, 830, and 840 are high-brightness colors such as white, the reflectance for the light output from the proximity sensor or color sensor may increase, and thus, the amount of light that has reflected from the patterns 810, 820, 830, and 840 and returned may increase. In a case in which the colors of each of the patterns 810, 820, 830, and 840 are low-brightness colors such as black, the reflectance for the light output from the proximity sensor or color sensor may decrease, and thus, the amount of light that has reflected from the patterns 810, 820, 830, and 840 and returned may decrease. Based on the amounts of light reflected from each of the patterns 810, 820, 830, and 840 being large, the at least one processor 340 may determine that the brightness of the colors of each of the patterns 810, 820, 830, and 840 is high. Based on the amounts of light reflected from the patterns 810, 820, 830, and 840 being small, the at least one processor 340 may determine that the brightness of the colors of the patterns 810, 820, 830, and 840 is low.

In an embodiment of the disclosure, the at least one processor 340 may detect the colors of each of the patterns 810, 820, 830, and 840 based on the amounts of light reflected from each of the patterns 810, 820, 830, and 840. The at least one processor 340 may identify the grade of the at least one filter 131 based on the detected colors of the patterns 810, 820, 830, and 840.

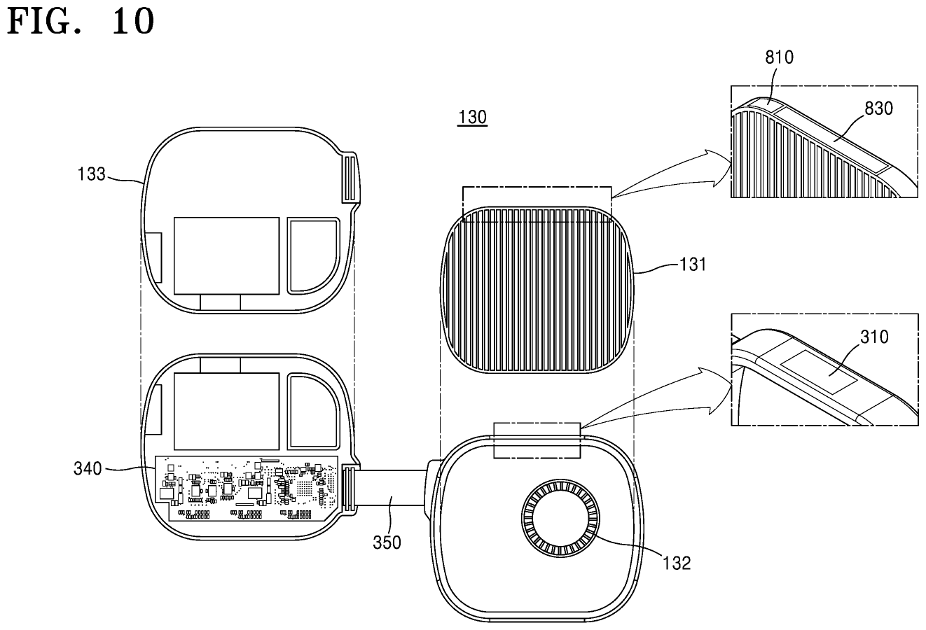

FIG. 10 is a diagram illustrating the clean air blowing module 130 of the electronic mask 100 according to an embodiment of the disclosure. The clean air blowing module 130 may include the at least one filter 131, the fan 132, and the case 133.

In an embodiment of the disclosure, the at least one filter 131 may be arranged inside the case 133. For example, the at least one filter 131 may be inserted into the case 133 to be adjacent to the inner surface of the case 133 on which the fan 132 is mounted.

In an embodiment of the disclosure, the at least one filter 131 may include patterns 810 and 830. The patterns 810 and 830 may be arranged on an edge region of the at least one filter 131. The patterns 810 and 830 may have a dot shape or a shape extending along the edge region of the at least one filter 131. Colors designated based on the grade of the at least one filter 131 may be applied to the patterns 810 and 830. For example, the inside of each of the patterns 810 and 830 may be colored with a designated color.

In an embodiment of the disclosure, the fan 132 may be mounted on the inner surface of the case 133. For example, the fan 132 may be mounted on the rear surface of the case 133. The fan 132 may blow air toward the at least one filter 131.

In an embodiment of the disclosure, the case 133 may form an outer edge of the clean air blowing module 130. The case 133 may fix the at least one filter 131 and the fan 132 such that the at least one filter 131 and the fan 132 are arranged in designated positions and directions. The case 133 may include the sensing module 310, the control board 340, and the connector 350.

In an embodiment of the disclosure, the sensing module 310 may be arranged on one surface of the case 133. For example, the sensing module 310 may be arranged on an upper edge of the case 133. The sensing module 310 may obtain sensing data related to the at least one filter 131. The sensing module 310 may obtain sensing data related to the colors of the patterns 810 and 830 of the at least one filter 131. The sensing data may include information about the amount of light reflected from the patterns 810 and 830. The sensing module 310 may include a proximity sensor or a color sensor. For example, the sensing module 310 may output light toward the patterns 810 and 830 and obtain sensing data based on the amount of light reflected from the patterns 810 and 830.

In an embodiment of the disclosure, the control board 340 may be arranged inside the case 133. For example, the control board 340 may be mounted on the inner surface of the case 133. The control board 340 may include at least one processor. The control board 340 may control the overall operation of the clean air blowing module 130 and the sensing module 310.

In an embodiment of the disclosure, when the case 133 is opened, the connector 350 may connect the left and right portions of the case 133 to each other. The connector 350 may electrically connect the control board 340 to the sensing module 310. For example, the connector 350 may include therein a wire that electrically connects the control board 340 to the sensing module 310.

In an embodiment of the disclosure, the at least one processor may obtain the amount of light reflected from each of the patterns 810 and 830 based on the sensing data. The at least one processor may detect the colors of each of the patterns 810 and 830 based on the amount of light reflected from the patterns 810 and 830. The at least one processor may identify the grade of the at least one filter 131 based on the colors of the patterns 810 and 830.

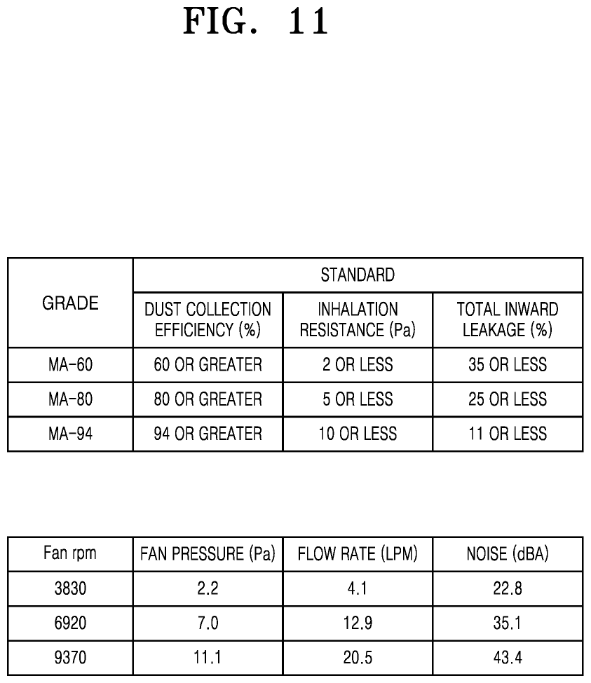

The grade of the filter 131 of the electronic mask 100 may be easily identified by using any of the methods described above. Hereinafter, a method of controlling the rotation speed of the fan 132 based on the grade of the filter 131 of the electronic mask 100 will be described with reference to FIG. 11.

FIG. 11 is a diagram illustrating control of the rotation speed of the fan 132 of the electronic mask 100 according to an embodiment of the disclosure.

In an embodiment of the disclosure, the inhalation resistance of the at least one filter 131 mounted on the electronic mask 100 may be determined according to the grade of the at least one filter 131. For example, in a case in which the grade of the at least one filter 131 is MA-60, the inhalation resistance may be about 2 Pa or less. For example, in a case in which the grade of the at least one filter 131 is MA-80, the inhalation resistance may be about 5 Pa or less. For example, in a case in which the grade of the at least one filter 131 is MA-94, the inhalation resistance may be about 10 Pa or less. Controlling the rotation speed of the fan 132 according to the grade of the at least one filter 131 mounted on the electronic mask 100 may help the user's breathing.

In an embodiment of the disclosure, the at least one processor 340 may control the rotation speed of the fan 132 based on the identified grade of the at least one filter 131. The at least one processor 340 may obtain, from a digital storage unit or memory, information about a pressure by the fan 132 according to the rotation speed of the fan 132. For example, in a case in which the rotation speed of the fan 132 is about 3830 RPM, the at least one processor 340 may obtain, from the storage unit, information indicating that the pressure of the fan 132 is about 2.2 Pa. For example, in a case in which the rotation speed of the fan 132 is about 6920 RPM, the at least one processor 340 may obtain, from the storage unit, information indicating that the pressure of the fan 132 is about 7.0 Pa. For example, in a case in which the rotation speed of the fan 132 is about 9370 RPM, the at least one processor 340 may obtain, from the storage unit, information indicating that the pressure of the fan 132 is about 11.1 Pa.

In an embodiment of the disclosure, the at least one processor 340 may control the rotation speed of the fan 132 such that the pressure by the fan 132 has a value similar to the inhalation resistance. For example, in a case in which the identified grade of the at least one filter 131 is MA-60, the at least one processor 340 may control the rotation speed of the fan 132 to be about 3830 RPM such that the pressure by the fan 132 is about 2.2 Pa to be similar to the inhalation resistance value of about 2 Pa. For example, in a case in which the identified grade of the at least one filter 131 is MA-80, the at least one processor 340 may control the rotation speed of the fan 132 to be about 6920 RPM such that the pressure by the fan 132 is about 7.0 Pa to be similar to the inhalation resistance value of about 5 Pa. For example, in a case in which the identified grade of the at least one filter 131 is MA-94, the at least one processor 340 may control the rotation speed of the fan 132 to be about 9370 RPM such that the pressure by the fan 132 is about 11.1 Pa to be similar to the inhalation resistance value of about 10 Pa.

In an embodiment of the disclosure, the at least one processor 340 may increase the rotation speed of the fan 132 as the dust collection efficiency related to the grade of the at least one filter 131 mounted on the electronic mask 100 increases. For example, the at least one processor 340 may increase the rotation speed of the fan 132 as the dust collection efficiency of the at least one filter 131 mounted on the electronic mask 100 increases. The at least one processor 340 may decrease the rotation speed of the fan 132 as the grade of the at least one filter 131 mounted on the electronic mask 100 is lower. For example, the at least one processor 340 may decrease the rotation speed of the fan 132 as the dust collection efficiency of the at least one filter 131 mounted on the electronic mask 100 decreases. For example, the at least one processor 340 may adjust the rotation speed of the fan 132 to be about 3830 based on the grade of the at least one filter 131 mounted on the electronic mask 100 being identified as the MA-60 grade, may adjust the rotation speed of the fan 132 to be about 6920 RPM based on the grade of the at least one filter 131 mounted on the electronic mask 100 being identified as the MA-80 grade, and may adjust the rotation speed of the fan 132 to be about 9370 RPM based on the grade of the at least one filter 131 mounted on the electronic mask 100 being identified as the MA-94 grade. Accordingly, the at least one processor 340 may automatically appropriately adjust the rotation speed of the fan 132 according to the grade of the at least one filter 131 mounted on the electronic mask 100. Accordingly, the electronic mask 100 according to the disclosure may improve a function of helping a user wearing the electronic mask 100 breathe comfortably, even when filters 131 of different grades are installed. In addition, the electronic mask 100 according to the disclosure may prevent unnecessary high-speed rotation of the fan 132 by adjusting the rotation speed of the fan 132 when filters 131 of different grades are mounted, thereby efficiently managing the usage time of the battery of the electronic mask 100.

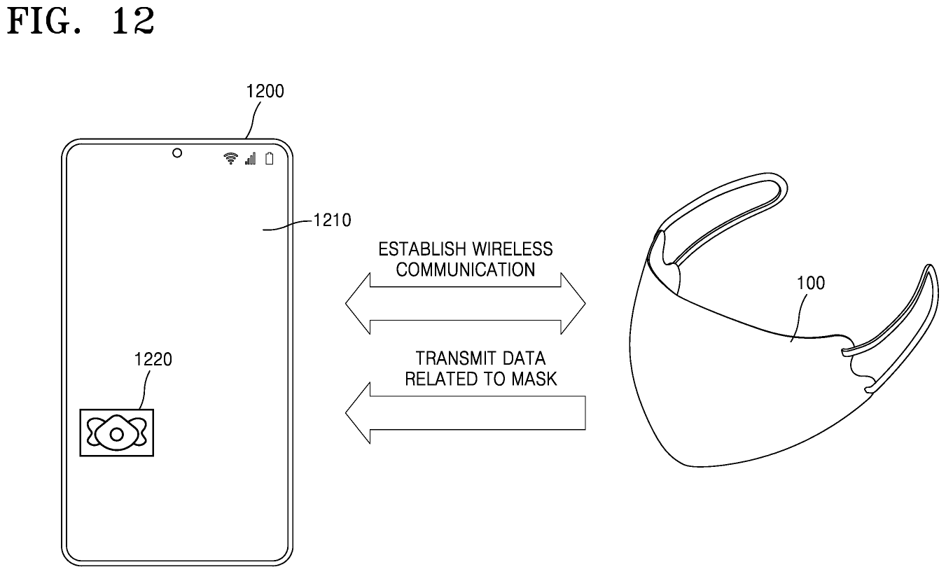

The at least one processor 340 of the electronic mask 100 may identify the grade of the at least one filter 131 mounted on the electronic mask 100, and control the rotation speed of the fan 132 based on the identified grade. By allowing the user to easily check data related to the electronic mask 100, such as information about the identified grade of the at least one filter 131, by using an external electronic device 1200, the convenience of the user wearing the electronic mask 100 may be improved. Hereinafter, the electronic mask 100 establishing a communication with the external electronic device 1200 and transmitting data will be described with reference to FIG. 12.

FIG. 12 is a diagram illustrating the electronic mask 100 and the external electronic device 1200, according to an embodiment of the disclosure. For example, the external electronic device 1200 may be a mobile terminal such as a smart phone.

In an embodiment of the disclosure, the electronic mask 100 may include a communication module supporting wireless communication. The communication module may be arranged on the control board 340 on which the at least one processor is arranged. The communication module may support short-range communication. For example, the communication module may support BLE communication.

In an embodiment of the disclosure, the at least one processor 340 may control the communication module to establish wireless communication with the external electronic device 1200. For example, when detecting that the user wears the electronic mask 100, the at least one processor 340 may control the communication module to establish wireless communication with the external electronic device 1200.

In an embodiment of the disclosure, the at least one processor 340 may control the communication module to transmit data related to the electronic mask 100, to the external electronic device 1200. The data related to the electronic mask 100 may be data related to the at least one filter 131 of the electronic mask 100. For example, the data related to the electronic mask 100 may be data related to the grade of the at least one filter 131 of the electronic mask 100. The data related to the electronic mask 100 may be data related to the rotation speed of the fan 132 of the electronic mask 100.

In an embodiment of the disclosure, the electronic mask 100 may communicate with the external electronic device 1200 such as a mobile terminal. The at least one processor 340 of the electronic mask 100 may transmit the data related to the electronic mask 100 to the external electronic device 1200 by using the communication module. For example, the at least one processor 340 may transmit, the external electronic device 1200, information about the grade of the at least one filter 131 mounted on the electronic mask 100, information about the rotation speed of the fan 132 of the electronic mask 100, information about the remaining power of the battery of the electronic mask 100, and information about the user's breath obtained through a breath sensor included in the electronic mask 100.

In an embodiment of the disclosure, the external electronic device 1200 may visually display the data related to the electronic mask 100, through a display 1210. For example, when wireless communication with the electronic mask 100 is established, the external electronic device 1200 may display an icon 1220 indicating that the data related to the electronic mask 100 may be displayed.

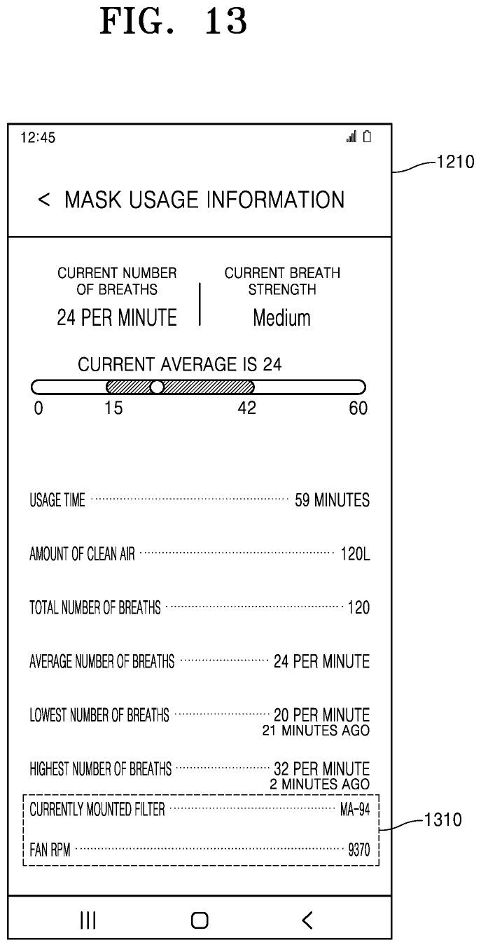

Hereinafter, a method of displaying data related to the electronic mask 100 on the display 1210 by the external electronic device 1200 having received the data related to the electronic mask 100 from the electronic mask 100 will be described with reference to FIGS. 13 to 15.

FIG. 13 is a diagram illustrating the external electronic device 1200 displaying information about the filter 131 of the electronic mask 100, according to an embodiment of the disclosure.

In an embodiment of the disclosure, the external electronic device 1200 may display data related to the electronic mask 100, on the display 1210. For example, the external electronic device 1200 may display, on the display 1210, first information 1310 about the grade of the filter 131 currently mounted on the electronic mask 100, and the rotation speed of the fan 132 of the electronic mask 100. For example, the external electronic device 1200 may display the current number of breaths per minute, real-time breath strength, the usage time of the electronic mask 100, the amount of clean air, the total number of breaths of the user, the total breaths of the user, the average number of breaths per minute of the user, the lowest number of breaths per minute of the user, and the highest number of breaths per minute of the user.

In an embodiment of the disclosure, the external electronic device 1200 may provide the user with information transmitted from the electronic mask 100, through a certain application. For example, the external electronic device 1200 may execute an application related to the electronic mask 100. The external electronic device 1200 may display an execution window of the application, on the display 1210. The external electronic device 1200 may display the first information 1310 about the grade of the filter 131 currently mounted on the mask, and the rotation speed of the fan 132, through the execution window of the application displayed on the display 1210. In addition, the external electronic device 1200 may display, through the execution window of the application displayed on the display 1210, the usage time, the amount of clean air, the total number of breaths, the average number of breaths per minute, the lowest number of breaths per minute, the highest number of breaths per minute, the current number of breaths per minute, the real-time breath strength, the remaining power of the battery, and the like.

In an embodiment of the disclosure, the external electronic device 1200 may display the information in the execution window of the application on the display 1210. The external electronic device 1200 may provide, in the execution window of the application on the display 1210, information about whether the currently mounted filter is suitable for the user's current situation, or information about whether the current breathing state is stable. Accordingly, by checking the information displayed in the execution window of the application, it is possible to check whether the currently mounted filter is suitable for the current situation, or whether the current breathing state is stable. For example, when the user is about to start exercising, the user may check information indicating that the filter 131 currently mounted on the electronic mask 100 is the MA-94 grade, through the execution window of the application on the display 1210 of the external electronic device 1200. In order to make breathing more comfortable during an exercise, the user may separate the filter 131 of the MA-94 grade from the case 133 of the electronic mask 100, and attach the filter 131 of the MA-60 grade to the electronic mask 100. When the filter 131 mounted on the electronic mask 100 is replaced, the external electronic device 1200 may display a notification that the filter 131 mounted on the electronic mask 100 has been replaced, through the execution window of the application on the display 1210. For example, the external electronic device 1200 may display the phrase “Filter replacement completed, MA-94->MA-60” through the execution window of the application on the display 1210.

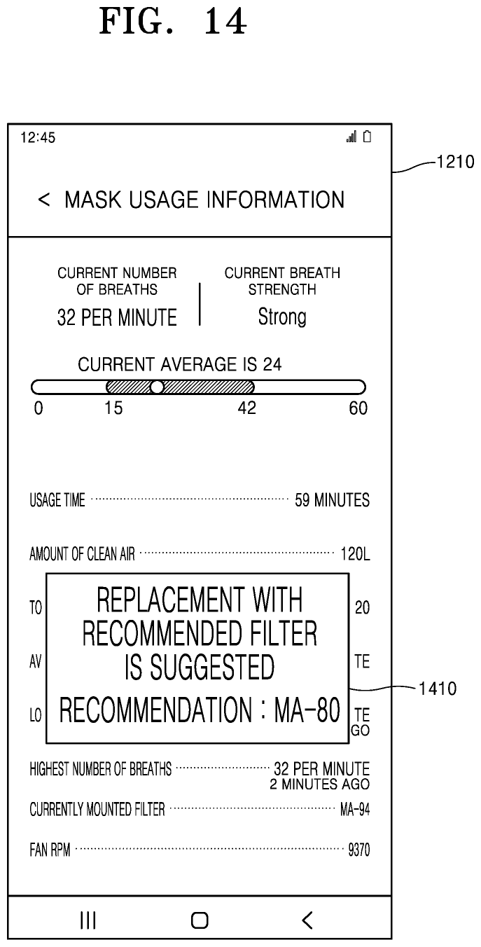

FIG. 14 is a diagram illustrating the external electronic device 1200 guiding a user to replace the filter 131 of the electronic mask 100, according to an embodiment of the disclosure.

In an embodiment of the disclosure, the external electronic device 1200 may display, on the display 1210, a first guide 1410 for suggesting replacement with the recommended filter 131, and indicating the grade of the recommended filter 131. Based on determining that the filter 131 currently mounted on the electronic mask 100 is unsuitable for the usage time, the amount of clean air, the total number of breaths, the average number of breaths per minute, the lowest number of breaths per minute, the highest number of breaths per minute, the current number of breaths per minute, the real-time breath strength, and the like, the external electronic device 1200 may recommend to replace the filter 131. The external electronic device 1200 may determine the grade of the recommended filter 131 according to the usage time, the amount of clean air, the total number of breaths, the average number of breaths per minute, the lowest number of breaths per minute, the highest number of breaths per minute, the current number of breaths per minute, the real-time breath strength, and the like.

In an embodiment of the disclosure, the external electronic device 1200 may recommend the type of the at least one filter 131 based on information about breathing of the user obtained through the breath sensor mounted on the electronic mask 100. The breath sensor mounted on the electronic mask 100 may measure the number of breaths per minute of the user, and the wind strength when the user exhales. For example, when the number of breaths per minute of the user measured by the breath sensor mounted on the electronic mask 100 increases or the wind strength increases, the external electronic device 1200 may determine that it is difficult for the user to breathe. In this case, the external electronic device 1200 may recommend the filter 131 having a lower dust collection efficiency and a lower grade than those of the filter 131 currently mounted on the electronic mask 100. For example, in a case in which the number of breaths per minute of the user increases to 32/min and the real-time breath strength (exhalation/inhalation strength) is changed to ‘strong’ in a state where it is confirmed that the filter 131 of the MA-94 grade is mounted on the electronic mask 100, the external electronic device 1200 may provide the first guide 1410 including a recommendation notification to replace the filter 131 of the MA-94 grade with the filter 131 of the MA-80 grade.

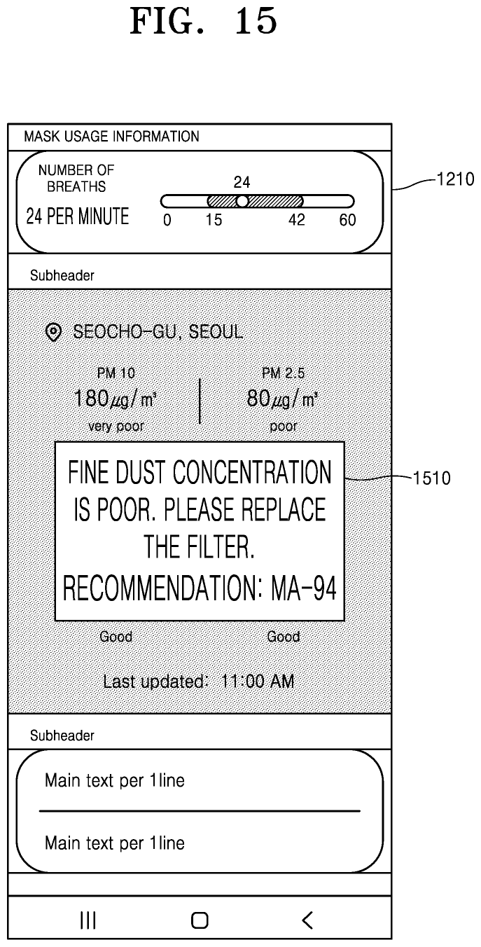

FIG. 15 is a diagram illustrating the external electronic device 1200 guiding a user to replace the filter 131 of the electronic mask 100, according to an embodiment of the disclosure.

In an embodiment of the disclosure, the external electronic device 1200 may display, on the display 1210, a second guide 1510 for suggesting to replace the filter 131 because the concentration of fine dust is poor, and indicating the grade of the recommended filter 131. The external electronic device 1200 may recommend replacement of the filter 131, based on determining that the filter 131 currently mounted on the electronic mask 100 is unsuitable for the amount of clean air. The external electronic device 1200 may determine the grade of the recommended filter 131 according to the amount of clean air.

In an embodiment of the disclosure, the external electronic device 1200 may obtain information related to air quality, such as the concentration of fine dust in the current location, from a weather server or the like. When the external electronic device 1200 receives information about the currently mounted filter 131 from the electronic mask 100, the external electronic device 1200 may determine whether the filter 131 of an appropriate grade is mounted, based on the concentration of fine dust in the air at the current location. When the mounted filter 131 is unsuitable for the concentration of fine dust in the air at the current location, the external electronic device 1200 may provide the second guide 1510 including a recommendation notification to replace the filter 131.

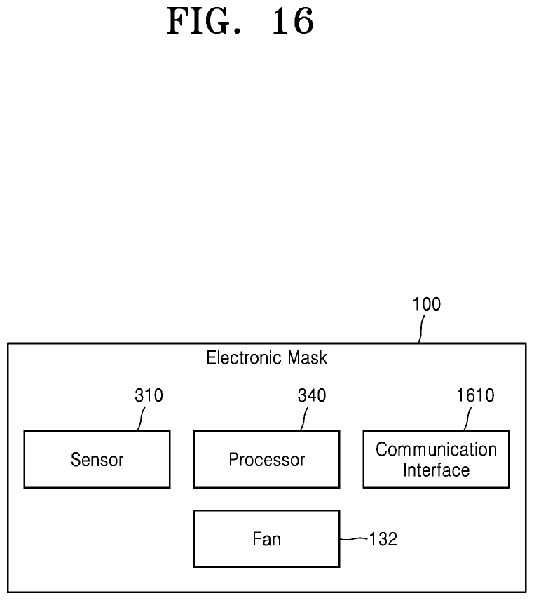

FIG. 16 is a block diagram illustrating an electronic mask according to an embodiment of the disclosure. The electronic mask 100 according to an embodiment of the disclosure may include at least one processor 340, at least one sensor 310, a communication interface 1610, and a fan 132.

The electronic mask 100 according to an embodiment of the disclosure may include the at least one filter 131, the sensing module 310 or 510 and 520 including at least one sensor and configured to obtain sensing data related to the at least one filter 131, the fan 132 that generates a flow of air toward the at least one filter 131, and the at least one processor 340. The at least one processor 340 may identify the grade of the at least one filter 131 based on the sensing data obtained from the sensing module 310 or 510 and 520. The at least one processor 340 may control the rotation speed of the fan 132 based on the grade of the at least one filter 131.

In an embodiment of the disclosure, the at least one processor 340 may control the sensing module 310 to detect a signal transmitted from the RFID tag 210 or 330 of the at least one filter 131. The at least one processor 340 may identify the grade of the at least one filter 131 based on the RF signal transmitted from the RFID tag 210 or 330.

In an embodiment of the disclosure, the RFID tag 210 or 330 may be arranged in an edge region of the at least one filter 131, and may be configured to transmit the signal at a signal frequency in a predesignated frequency range. The sensing module 310 may be arranged within a recognition distance of the RFID tag 210 or 330, the recognition distance being determined based on the predesignated frequency range.

In a case in which the signal frequency is in a first frequency range between about 1 Hz and about 10 kHz, the sensing module 310 may be arranged adjacent to the RFID tag 210 or 330 to face the edge region of the at least one filter 131. In a case in which the signal frequency is in a second frequency range higher than the first frequency range, between about 1 MHz and about 10 GHz, the sensing module 310 may be arranged on a control board 340, which includes the at least one processor, to be spaced apart from the RFID tag 210 or 330.

In an embodiment of the disclosure, the sensing module may include a plurality of sensors 510 and 520. The at least one processor 340 may detect the shape of the at least one filter 131 by obtaining a shape detection value related to the at least one filter 131 from the plurality of sensors 510 and 520. The at least one processor 340 may identify the grade of the at least one filter 131 based on the shape of the at least one filter 131.

In an embodiment of the disclosure, the plurality of sensors 510 and 520 may include the first sensor 510 arranged in a first corner region of the at least one filter 131 and configured to detect the shape of the at least one filter 131 in a first direction. The plurality of sensors 510 and 520 may include the second sensor 520 arranged in a second corner region of the at least one filter 131 and configured to detect the shape of the at least one filter 131 in a second direction.

In an embodiment of the disclosure, the at least one processor 340 may detect the color of the at least one filter 131 by obtaining a color detection value related to the at least one filter 131 from the sensing module 310. The at least one processor 340 may identify the grade of the at least one filter 131 based on the color of the at least one filter 131.

In an embodiment of the disclosure, the color may be applied to the at least one pattern 810 and 830 arranged on an edge region of the at least one filter 131. The sensing module 310 may output light toward the at least one pattern 810 and 830. The sensing module 310 may detect the color by detecting a reflected amount of the light.

In an embodiment of the disclosure, the at least one processor 340 may increase the rotation speed of the fan 132 as the dust collection efficiency related to the grade of the at least one filter 131 mounted on the electronic mask 100 increases.

The electronic mask 100 according to an embodiment of the disclosure may include a communication module supporting wireless communication. The at least one processor 340 may control the communication module to establish wireless communication with the external electronic device 1200. The at least one processor 340 may further control the communication module to transmit data related to the electronic mask 100 to the external electronic device 1200.

In an embodiment of the disclosure, the data related to the electronic mask 100 may include the grade of the at least one filter 131 and the rotation speed of the fan 132.

A method of controlling the electronic mask 100 including the at least one filter 131, the sensing module 310 or 510 and 520 configured to obtain sensing data related to the at least one filter 131, and the fan 132 configured to generate a flow of air toward the at least one filter 131 according to an embodiment of the disclosure may include identifying the grade of the at least one filter 131 based on the sensing data obtained from the sensing module 310 or 510 and 520. The method of controlling the electronic mask 100 according to an embodiment of the disclosure may include controlling the rotation speed of the fan 132 based on the grade of the at least one filter 131.

In an embodiment of the disclosure, the identifying of the grade of the at least one filter 131 may include detecting a signal transmitted from the RFID tag 210 or 330 of the at least one filter 131. The identifying of the grade of the at least one filter 131 may include identifying the grade of the at least one filter 131 based on the signal transmitted from the RFID tag 210 or 330.

In an embodiment of the disclosure, the identifying of the grade of the at least one filter 131 may include detecting the shape of the at least one filter 131 by obtaining a shape detection value related to the at least one filter 131 from the plurality of sensors 510 and 520. The identifying of the grade of the at least one filter 131 may include identifying the grade of the at least one filter 131 based on the shape of the at least one filter 131.

In an embodiment of the disclosure, the identifying of the grade of the at least one filter 131 may include detecting the color of the at least one filter 131 by obtaining a color detection value related to the at least one filter 131 from the sensing module 310. The identifying of the grade of the at least one filter 131 may include identifying the grade of the at least one filter 131 based on the color of the at least one filter 131.

In an embodiment of the disclosure, the color may be applied to the at least one pattern 810 and 830 arranged on an edge region of the at least one filter 131. The detecting of the color of the at least one filter 131 may include outputting light toward the at least one pattern 810 and 830. The detecting of the color of the at least one filter 131 may include detecting the color by detecting a reflected amount of the light.

In an embodiment of the disclosure, the controlling of the rotation speed of the fan 132 may include increasing the rotation speed of the fan 132 as the dust collection efficiency related to the grade of the at least one filter 131 mounted on the electronic mask 100 increases.

The method of controlling the electronic mask 100 according to an embodiment of the disclosure may include establishing wireless communication with the external electronic device 1200. The method of controlling the electronic mask 100 according to an embodiment of the disclosure may include transmitting data related to the electronic mask 100 to the external electronic device 1200.

In an embodiment of the disclosure, the data related to the electronic mask 100 may include the grade of the at least one filter 131 and the rotation speed of the fan.

The method according to an embodiment of the disclosure may be embodied as program commands executable by various computer devices, and recorded on a computer-readable medium. The computer-readable medium may include program commands, data files, data structures, or the like separately or in combinations. The program commands to be recorded on the medium may be specially designed and configured for the disclosure or may be well-known to and be usable by those skill in the art of computer software. Examples of the computer-readable recording medium include magnetic media such as hard disks, floppy disks, or magnetic tapes, optical media such as a compact disc read-only memory (CD-ROM) or a digital video disc (DVD), magneto-optical media such as a floptical disk, and hardware devices such as ROM, random-access memory (RAM), or flash memory, which are specially configured to store and execute program commands. Examples of the program commands include not only machine code, such as code made by a compiler, but also high-level language code that is executable by a computer by using an interpreter or the like.

An embodiment of the disclosure may be implemented as a recording medium including computer-readable instructions such as a computer-executable program module. The computer-readable medium may be any available medium which is accessible by a computer, and may include a volatile or non-volatile medium and a removable or non-removable medium. Also, the computer-readable medium may include a computer storage medium and a communication medium. The computer storage media include both volatile and non-volatile, removable and non-removable media implemented in any method or technique for storing information such as computer readable instructions, data structures, program modules or other data. The communication media typically include computer-readable instructions, data structures, program modules, other data of a modulated data signal, or other transmission mechanisms, and examples thereof include an arbitrary information transmission medium. Also, an embodiment of the disclosure may be implemented as a computer program or a computer program product including computer-executable instructions such as a computer program executed by a computer.

A machine-readable storage medium may be provided in the form of a non-transitory storage medium. Here, the term ‘non-transitory storage medium’ refers to a tangible device and does not include a signal (e.g., an electromagnetic wave), and the term ‘non-transitory storage medium’ does not distinguish between a case where data is stored in a storage medium semi-permanently and a case where data is stored temporarily. For example, the non-transitory storage medium may include a buffer in which data is temporarily stored.

According to an embodiment of the disclosure, the methods according to an embodiment of the disclosure may be included in a computer program product and then provided. The computer program product may be traded as commodities between sellers and buyers. The computer program product may be distributed in the form of a machine-readable storage medium (e.g., a CD-ROM, or may be distributed online (e.g., downloaded or uploaded) through an application store or directly between two user devices (e.g., smart phones). In a case of online distribution, at least a portion of the computer program product (e.g., a downloadable app) may be temporarily stored in a machine-readable storage medium such as a manufacturer's server, an application store's server, or a memory of a relay server.

1. An electronic mask comprising:
at least one filter;
at least one sensor configured to obtain sensing data related to the at least one filter;
a fan configured to generate a flow of air toward the at least one filter; and
at least one processor configured to:
identify a grade of the at least one filter based on the sensing data obtained from the at least one sensor, and
control a rotation speed of the fan based on the grade of the at least one filter.

2. The electronic mask of claim 1, wherein the at least one processor is further configured to:
control the at least one sensor to detect a signal transmitted from a radio-frequency identification (RFID) tag of the at least one filter, and
identify the grade of the at least one filter based on the signal transmitted from the RFID tag.

3. The electronic mask of claim 2, wherein the RFID tag is provided on an edge region of the at least one filter, and is configured to transmit the signal at a signal frequency in a predesignated frequency range, and
wherein the at least one sensor is provided within a recognition distance of the RFID tag, the recognition distance being determined based on the predesignated frequency range.

4. The electronic mask of claim 3, wherein the signal frequency is between about 1 Hz and about 10 kHz, and
wherein the at least one sensor is adjacent to the RFID tag to face the edge region of the at least one filter.

5. The electronic mask of claim 3, wherein the signal frequency is between about 1 MHz and about 10 GHz, and
wherein the at least one sensor is provided on a control board to be spaced apart from the RFID tag, the control board comprising the at least one processor.

6. The electronic mask of claim 1, wherein the at least one sensor comprises a plurality of sensors, and
wherein the at least one processor is further configured to:
detect a shape of the at least one filter by obtaining a shape detection value related to the at least one filter from the plurality of sensors, and
identify the grade of the at least one filter based on the shape of the at least one filter.

7. The electronic mask of claim 6, wherein the plurality of sensors comprise:
a first sensor provided in a first corner region of the at least one filter and configured to detect the shape of the at least one filter in a first direction, and
a second sensor provided in a second corner region of the at least one filter and configured to detect the shape of the at least one filter in a second direction.

8. The electronic mask of claim 1, wherein the at least one processor is further configured to:
detect a color of the at least one filter by obtaining a color detection value related to the at least one filter from the at least one sensor, and
identify the grade of the at least one filter based on the color of the at least one filter.

9. The electronic mask of claim 8, wherein the color is applied to at least one pattern arranged on an edge region of the at least one filter, and
wherein the at least one sensor is further configured to:
output light toward the at least one pattern, and
detect the color by detecting a reflected amount of the light.

10. The electronic mask of claim 1, wherein the at least one processor is further configured to increase the rotation speed of the fan as a dust collection efficiency related to the grade of the at least one filter mounted on the electronic mask increases.

11. The electronic mask of claim 1, further comprising a communication interface configured to support wireless communication,
wherein the at least one processor is further configured to control the communication interface to:
establish wireless communication with an external electronic device, and
transmit data related to the electronic mask to the external electronic device.

12. The electronic mask of claim 11, wherein the data related to the electronic mask comprises the grade of the at least one filter and the rotation speed of the fan.

13. A method of controlling an electronic mask comprising at least one filter, at least one sensor configured to obtain sensing data related to the at least one filter, and a fan configured to generate a flow of air toward the at least one filter, the method comprising:
identifying a grade of the at least one filter based on the sensing data obtained from the at least one sensor; and
controlling a rotation speed of the fan based on the grade of the at least one filter.

14. The method of claim 13, wherein the identifying of the grade of the at least one filter comprises:
detecting a signal transmitted from a radio-frequency identification (RFID) tag of the at least one filter; and
identifying the grade of the at least one filter based on the signal transmitted from the RFID tag.

15. The method of claim 13, wherein the at least one sensor comprises a plurality of sensors, and
wherein the identifying of the grade of the at least one filter comprises:
detecting a shape of the at least one filter by obtaining a shape detection value related to the at least one filter from the plurality of sensors; and
identifying the grade of the at least one filter based on the shape of the at least one filter.

16. The method of claim 13, wherein the identifying of the grade of the at least one filter comprises:
detecting a color of the at least one filter by obtaining a color detection value related to the at least one filter from the at least one sensor; and
identifying the grade of the at least one filter based on the color of the at least one filter.

17. The method of claim 16, wherein the color is applied to at least one pattern arranged on an edge region of the at least one filter, and
wherein the detecting of the color of the at least one filter comprises:
outputting light toward the at least one pattern, and
detecting the color by detecting a reflected amount of the light.

18. The method of claim 13, wherein the controlling of the rotation speed of the fan comprises increasing the rotation speed of the fan as a dust collection efficiency related to the grade of the at least one filter mounted on the electronic mask increases.

19. The method of claim 13, further comprising:
establishing wireless communication with an external electronic device through a communication interface of the electronic mask; and
transmitting data related to the electronic mask to the external electronic device through the communication interface.

20. The method of claim 19, wherein the data related to the electronic mask comprises the grade of the at least one filter and the rotation speed of the fan.
